(19) United States
(12) Patent Application Publication
(10) Pub. No.: US 2022/0189642 A1
(43) Pub. Date: Jun. 16, 2022

(54) SOCIAL DISTANCING OPTIMIZATION

(71) Applicant: International Business Machines Corporation, Armonk, NY (US)

(72) Inventors: Shunguo Yan, Austin, TX (US); Andrew J. Lavery, Austin, TX (US); Su Liu, Austin, TX (US); Jonathan D. Dunne, Dungarvan (IE)

(21) Appl. No.: 17/120,545

(22) Filed: Dec. 14, 2020

(51) Int. Cl. G16H 50/80; G06F 17/18; G06F 17/16

(52) U.S. Cl. CPC G16H 50/80; G06F 17/16; G06F 17/18

(57) ABSTRACT

A processor may receive congregation data. The congregation data may be associated with a particular area. The processor may analyze the congregation data. The processor may generate a social movement model of the particular area. The processor may generate one or more routes from a first location to a second location in the particular area based on the social movement model. The one or more routes may reduce the level of exposure to an infectious disease.

### BACKGROUND

The present disclosure relates generally to the field of epidemiology, and more particularly to social distancing during an infectious outbreak.

The field of epidemiology studies the incidence, distribution, and possible control of diseases and other related health factors. Contact tracing of those affected with an infectious disease (e.g., COVID-19) can play a powerful role in controlling and preventing the spread of the disease. Current methods of contract tracing often involve intensive work of persons in the healthcare field to determine if a person is a risk to others he came into contact with. Often medical advice regarding how to avoid becoming ill with an infectious disease can include getting a vaccine (e.g., a flu vaccine) and social distancing. However, a vaccine might not always be available for a particular infectious disease and social distancing can be difficult as some particular activities can require or result in a breach of social distancing. In addition, the effectiveness of social distancing is often determined by community participation.

### SUMMARY

Embodiments of the present disclosure include a method, computer program product, and system for reducing a level of exposure of contact with an infectious disease.

A processor may receive congregation data. The congregation data may be associated with a particular area. The processor may analyze the congregation data. The processor may generate a social movement model of the particular area. The processor may generate one or more routes from a first location to a second location in the particular area based on the social movement model. The one or more routes may reduce the level of exposure to an infectious disease.

The above summary is not intended to describe each illustrated embodiment or every implementation of the present disclosure.

### BRIEF DESCRIPTION OF THE DRAWINGS

The drawings included in the present disclosure are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 illustrates an exemplary embodiment of a social distancing optimization system, in accordance with embodiments of the present disclosure.

FIG. 2 illustrates a flowchart of a method for social distancing optimization, in accordance with embodiments of the present disclosure.

FIG. 3A illustrates a cloud computing environment, in accordance with embodiments of the present disclosure.

FIG. 3B illustrates abstraction model layers, in accordance with embodiments of the present disclosure.

FIG. 4 illustrates a high-level block diagram of an example computer system that may be used in implementing one or more of the methods, tools, and modules, and any related functions, described herein, in accordance with embodiments of the present disclosure.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the disclosure.

### DETAILED DESCRIPTION

Aspects of the present disclosure relate generally to the field of epidemiology, and more particularly to social distancing during an infectious outbreak. Infectious diseases can have various levels of contagiousness and can infect people in a variety of ways (e.g., vectors, airborne, or direct contact diseases). One method of minimizing the spread of an infectious disease is to adhere by social distancing guidelines provided by public health officials. The rationale behind social distancing is to minimize the probability that a given healthy, uninfected person will come into contact with an infected person, thereby reducing or eliminating the possibility of the uninfected person becoming infected by that infected person. When adhered to, social distancing has proven to suppress community transmission and reduce the number of hospitalizations and/or deaths from the infectious disease.

Despite its effectiveness, social distancing can considerably impact and effect how a person interacts in a community. Though people may want to adhere by social distancing guidelines, in some situations, it can be difficult or impossible to be sufficiently separated from people in public spaces. These public spaces or areas include, but are not limited to gas stations, public transportation, pharmacies, and grocery stores. In order to be successful, social distancing often requires comprehensive and dedicated planning and implementation by the people within a community. However, such planning and implementation can be unsuccessful without social distancing information regarding the public space people need to interact with (e.g., maximum number of people at a service station). While people can make general decisions based on their previous experiences (e.g., regarding times when a service station or other public space is servicing a high number or low number of people), such generalizations can often be inaccurate. In these situations, a person may have to make a choice to either breach social distancing guidelines, and chance contact with an infected person, or have to reschedule their visit for another time. Providing social distancing information to people allows them to properly plan when and how they might enter a public space while attempting to socially distance.

For example, a person social distancing in their home may need to pick up their pharmaceutical prescription from a pharmacy. This person could generally plan to walk to pick up their prescription at the pharmacy during non-peak hours, (e.g., when the majority of people are working) to minimize their risk. In this example, while walking to the pharmacy during a non-peak time, the person may not be able to avoid other people or crowds walking in the same space and, may upon arrival at the pharmacy, find that the pharmacy is busy. As stated above, the person will likely have to make a choice if he/she wants to increase their level of exposure by waiting in a full pharmacy and breaching social distancing guidelines or walking back to their home using the busy sidewalks. Neither option is ideal and the person's possible level of exposure or contact with an infected person has already increased despite whatever previous prevention methods he may have taken. As a result, there is a need in the public health field for a method, which is disclosed herein, that can provide people with a means of determining and mitigating their level of exposure to an infectious disease and enable them to make decisions that are right for them based on their various circumstances.

In embodiments discussed herein, provided are solutions in the form of a method, system, and computer program product for optimizing social distancing. In embodiments, such solutions may be implemented in one or more software applications located on a person's smart device (e.g., mobile phone and/or computer) and which the person (e.g., user) has opted-in to provide data (e.g., location data, etc.). In some embodiments, a processor may receive congregation data associated with a particular area. In some embodiments, the processor analyze, statistically, the congregation data. In these embodiments, a processor may generate a social movement model of the particular area. In embodiments, a processor may generate one or more routes from a first location to a second location in the particular area based on the social movement model. In these embodiments, the one or more routes may reduce the level of exposure to the infectious disease. In some embodiments, the congregation data may be received from one or more devices associated with (e.g., belonging to) one or more users in the particular area.

For example, a person can be situated in their home (e.g., first location or starting position) and need to travel to a grocery store (e.g., second location or ending position) within a particular area. A particular area can include any bounded area that includes at least the first location and the second location. A person or user can use any device or number of devices (e.g., smart device and/or computer) to input their starting position and ending position. In embodiments, the processor can generate a weighted social movement model that models random movements and occurrences of people within a particular area. Such embodiments allow for modeling of people within a particular area and provides one or more routes or optimal paths for a person to travel from the first location (e.g., home) to the second location (e.g., grocery store) with the least amount of social interaction or contact with other people (e.g., social distancing).

The processor, via the device, can be configured to receive congregation data associated with the particular area. In embodiments, congregation data can include, but is not limited to, movement data (e.g., the movement of different people in a particular area), interarrival time (e.g., distance and time between the occurrences of two or more events), meeting times, encounter data, service data, wait-time data, and travel data. Congregation data can be collected in a variety of ways. For example, congregation data can be derived from GPS information collected from mobile devices (e.g., smart watch or mobile phone) of one or more people who are located in the particular area. In some embodiments, one or more IoT devices or sensors may be positioned within the bounded area to detect the some or all of the aforementioned types of congregation data.

In embodiments, the processor can receive congregation data from a historical repository of congregation data associated with a particular area, can be configured to use congregation data of an area having similar characteristics to the particular area (e.g., area having a similar population density and similar services), or any combination thereof. In embodiments, a processor can receive congregation data associated with real-time feed inputs associated with one or more devices of one or more people in a particular area. While in some embodiments, a processor can receive congregation data from each person located in a particular area, in other embodiments, a processor receives congregation data from less than all of the people located in a particular area. In some embodiments, a processor can receive regional data associated with the particular area. Regional data can include community scheduling information, such as music events, service sales, or any other community events, within the particular area that could affect how one or more people interact with the various public spaces in a particular area.

In embodiments, the processor may analyze, statistically, the congregation data to generate a social movement model. In these embodiments, a processor may construct a sparsity grid of the particular area. While various methods of defining a sparsity grid exist, one such method could include using a Voronoi Function. In embodiments, the Voronoi Function used to generate the sparsity grid can produced using a Fortunes Algorithm and a Bowyer-Watson Algorithm. A sparsity grid is a mathematical construct that allows for observation of different factors at various locations within a bounded area (e.g., the particular area of interest). For example, if a particular area of interest was suburb A, a processor could use movement data, interarrival times, and/or meeting times (e.g., people moving within this particular area) to generate a sparsity grid. From the sparsity grid, the processor may deduce what particular locations or regions within Suburb A have the greatest confluence of people at any given time of day. For example, there may be a greater confluence at a restaurant (e.g., public space) during traditional mealtimes and a lower population or confluence of people at the restaurant during non-mealtimes.

In embodiments, the processor may perform further statistical analyses using the congregation data. In these embodiments, the processor may be configured to perform one or more queue analyses using the congregation data. In embodiments, a processor may receive encounter data and meeting data (e.g., congregation data) and perform one or more queue analyses to determine and/or predict how many people/customers are processed in a specific time frame, the average delay of a person/customer, and the length of the waiting line within a particular area. When queue lines are long, and people are waiting for a service in a localized area, adhering social distancing guidelines can be difficult.

In embodiments, the processor may perform further statistical analyses using the congregation data. In these embodiments, the processor may be configured to perform a random walk analysis using previously received travel data (e.g., congregation data). In embodiments, a processor can determine one or more random walks or directions that a person might travel from the first location to the second location. For example, a random walk could include any possible path or set of directions a person, walking, using a motor vehicle, using public transportation routes, or any combination thereof, to travel from a first location (e.g., home) to a second location (e.g., grocery store). In most embodiments, the determined one or more random walks do not consider social distancing guidelines and can include even unlikely routes between a first location and a second location. For example, one generated random walk could include the shortest path between the two points while a second path could be a circuitous route having a distance significantly longer than the shortest path. In embodiments, results arising from various aforementioned statistical analyses can form a set of input feeds. For example, a set of input feeds can include the data results from the sparsity grid analysis, the queue analysis, and the random walk analysis.

In embodiments, a processor can produce a matrix using a set of input feeds to generate a social movement model. A processor can produce a matrix by using the set of input feeds and folding the data from the input feeds into a matrix using various techniques (e.g., matrix multiplication) to generate the social movement model. In some embodiments, the social movement model is generated by overlaying the resulting matrix data over a base sparsity grid. Using a social movement model, the processor can generate, based on social distancing guidelines, one or more routes from a first location to a second location. In embodiments, the one or more routes are particular random walks where if a person were to follow, would most likely meet or come into contact with the fewest number of people (e.g., weighted random walks).

In embodiments, congregation data may also include cohort data. Cohort data can include information regarding one or more groups of people that a person or user knows and is familiar with. Various public health studies have shown the longer an uninfected person remains in contact with an infected person the higher the risk the uninfected will become infected with the infectious disease. Traditionally, if a person sees another person that he knows while in a public space, it would be customary for the two people to stop and talk or chat. While under normal circumstances (e.g., when social distancing is not needed to mitigate an infectious disease) chatting with a friend in public is not a problem, in circumstances where social distancing is recommended or required, encountering and talking with a friend or person you know in public can result in not only contact with a person that likely breaches social distancing guidelines, but also increased duration of the contact that can increase a person's chance of becoming infected with the infectious disease. As such, to further minimize and reduce the level of exposure to the infectious disease, in some embodiments, a processor may use cohort data to determine the one or more routes a person could take from the first location to a second location. For example, a processor could receive cohort data that indicates that a friend of a person wanting to travel from their home to a grocery store is likely to intersect one of the suggested routes at a particular time. In this example, the processor could consider this information and provide the person and the friend with an alternative route that adheres to the social distancing guidelines.

In some embodiments, a processor may generate cohort data by compiling a person's contact data (e.g., instant messages, text messages, and call logs) to determine how well known the two people know each other. For example, if two people exchange a high number of text messages and calls each day, a processor may determine that the two people know each other well. In this example, a processor may deduce that if these two people were to be in a similar location, the two people would be likely to stop and talk. Alternatively, if the two people barely interact, a processor may determine that the two people do not know each other very well and are unlikely to interact. In some embodiments, while a processor might receive cohort data, the processor can infer from the contact data (e.g., level of observed communication patterns), whether it is likely the two people will likely want to communicate in the public space for a particular duration. As such, even if a person known to the person traveling from the first location to the second location may intersect one of the suggested routes, a processor may still provide the particular route if the processor determines the person traveling is unlikely to jeopardize one another's social distancing preferences (e.g., as based on the guidelines).

In some embodiments, a processor can receive the opt-in information from each person associated with the cohort data that may be collected from a mobile device. Alternatively, in some embodiments, cohort data may be collected from a plethora of sources. In these embodiments, cohort data can be based on historical behaviors a processor observes from the person traveling from first location to second location. For example, if a person traveling during a previous occasion came into contact with a person he knows and stopped to talk, a processor could measure the duration of the conversation as well as how close each person was to each other (e.g., was each person separated by at least 6 feet) to determine if a breach in the social distancing guidelines occurred. In these embodiments, such information could be collected by the processor via the person's smart device. Such options can be configured by a person or user to determine how strictly a processor should adhere to social distancing guidelines when generating one or more routes that will reduce the level of exposure. As discussed herein, depending on the infectious disease of concern, social distancing guidelines may be different. For example, while some public health officials recommend distancing between people to be at least 6 feet for some infectious diseases, others recommend people to be separated by at least 3 feet. As such, a processor may be configured to receive this information from a central source of infectious disease information (e.g., Center for Disease Control), or may alternatively be configured to receive inputs from a person or user regarding what the social distancing guideline are.

In some embodiments, a processor may generate one or more routes from a first location to a second location in the particular area based on the social movement model. In some embodiments, a processor can generate a predicted level of exposure for each of the one or more routes. Depending on the generated route, there could be varying levels of exposure a person might come into contact with. For example, one route might predict that a person traveling might be exposed to two people while he travels the route, while another route may predict that the person traveling might be exposed to 10 people. As such, a predicted level of exposure may be compiled for each of the one or more generated routes. These embodiments allow a person traveling, to make the most knowledgeable decision based on what is best for them. In one example embodiment, a processor may generate three different routes (Route A, Rout B, and Route C) from a first location to a second location. In this example embodiment, Route A can be the shortest route, but with a high predicted level of exposure (e.g., possible contact with 100 people), Route B, could have a convoluted and lengthy route that is significantly longer that Route A with a low predicted level of exposure (e.g., possible contact with 0 people), and Route C can be a medium distanced route with a medium to low predicted level of exposure (e.g., possible contact with 2 people).

In this example embodiment, a person can choose which route would be best for him and his needs. For example, if time is not an issue of concern for a person traveling, a person might choose Route A (e.g., the safest, longest route) or Route B (that has a low level of predicted exposure), but if time is a concern (e.g., if there is an emergency), then the person could choose Route A. In some embodiments, a person can further decide the one or more generated routes of a processor by selecting options in a preferred filter option. A preferred filter option can include, but is not limited to, options such as shortest distance to travel and shortest travel time. A preferred filter option may be indicated by a person or user. Once indicated, a processor can filter the generated one or more routes to ensure that the routes generated are more likely than not to meet with the person or user's desires and social distancing needs.

In some embodiments, a processor can generate a predicted level of exposure or risk and an actual level of exposure or risk. For example, as discussed herein a processor predicts the level of exposure a person might be in contact with while traveling form the first location to the second location. As discussed herein, the processor can generate these predictions using various congregation data received both in real-time and congregation data collected and stored in a historical repository. While such data is helpful in making such predicted exposure, in some instances, a person or user may encounter a higher level of exposure than that initially predicted by the processor.

For example, a person may determine to walk a particular route generated by the processor to have a low level of exposure, but if while the person is traveling, the encounter a block festival, that was not previously considered by the processor, on a street he need to traverse in order to complete their route. If the person decides to continue on their route, the person is likely to have an increased level of exposure than previously predicted. In embodiments, a processor can be configured to receive actual contact data while traveling. In these embodiments, the processor can collect actual contact data while the person is traveling the particular route (e.g., via a smart device). The actual contact data can include, but is not limited to, how many people the person traveling was in contact with, how near other people were to him, and the duration of the contact to determine. In embodiments, when the person completes the route, the processor may generate an actual level of exposure of the infectious disease. Such embodiments can be used to generate future routes as well as allow the person to determine if self-isolation is necessary.

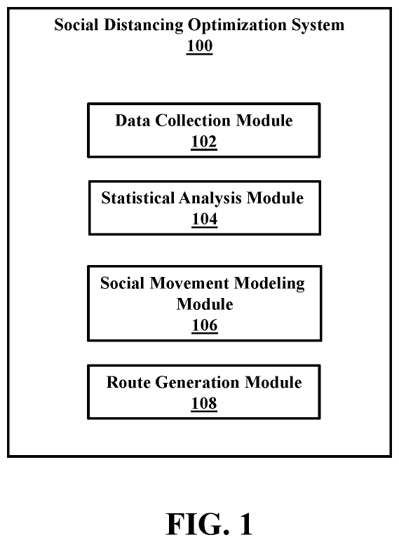

Referring now to FIG. 1, a block diagram of a social distancing optimization system 100, is depicted in accordance with embodiments of the present disclosure. FIG. 1 provides an illustration of only one implementation and does not imply any limitations with regard to the environments in which different embodiments may be implemented. Many modifications to the depicted environment may be made by those skilled in the art without departing from the scope of the invention as recited by the claims.

In embodiments, social distancing optimization system 100 can include data collection module 102, statistical analysis module 104, social movement modeling module 106, and route generation module 108, which are configured to be in communication with each other. In embodiments, data collection module 102 can be configured to receive congregation data associated with a particular area and from one or more devices belonging to one or more people in the particular area. Data collection module 102 can analyze the various congregation data and determine movement data, interarrival times, meeting times, encounter data, regional data (e.g., town and city data of the particular area), and travel data.

In embodiments, statistical analysis module 104 can analyze the congregation data. In some embodiments, statistical analysis module 104 can generate a sparsity grid using Voronoi Functions based on Fortunes Algorithms and Bowyer-Watson Algorithms using the movement data, interarrival times, meeting times, and regional data. In embodiments, statistical analysis module 104 can be configured to perform queue analyses using encounter and meeting data. In embodiments, statistical analysis module 104 can be configured to perform random walk analysis using travel data and regional data. In embodiments, data results derived results from sparsity grid, queue analysis, and random walk analysis can be a set of input feeds.

In embodiments, social movement modeling module 106 can be configured to generate a social movement model of the particular area. In embodiments, social movement modeling module 106 can be configured to generate a matrix by compiling a set of input feeds and processing the matrix (e.g., using matrix multiplication). In these embodiments, a social movement model can be generated by overlaying the processed matrix over the sparsity grid. The generate social movement model may allow a processor to view where people within the particular are likely to congregate at particular times.

In embodiments, route generation module 108 may generate one or more routes from a first location to a second location in the particular area based on the social movement model. In these embodiments, the one or more routes (e.g., weighted random walks) may be generated using the social movement model (e.g., demonstrating population distributions in a particular area) may reduce the level of exposure to the infectious disease my allowing a person to be aware of the likely environment of a particular area and allowing that person to make an educated decision about the level of exposure the person may endure while traveling from a first location to a second location. In some embodiments, the social distancing optimization system 100, utilizing the route generation module 108 may display the one or more routes to a user/person.

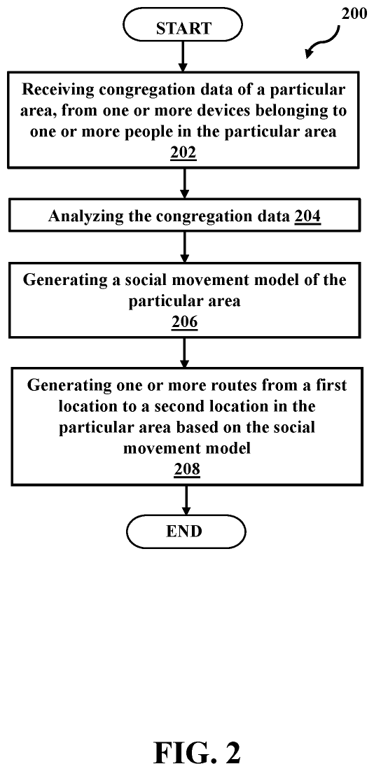

Referring now to FIG. 2, a flowchart illustrating an example method 200 for reducing a level of exposure of contact with an infectious disease, in accordance with embodiments of the present disclosure. In some embodiments, the method 200 begins at operation 202 where a processor receives congregation data. In these embodiments, congregation data may be associated with a particular area. In some embodiments, the congregation data may be collected from one or more devices associated with/belonging to one or more people in the particular area.

In some embodiments, the method 200 proceeds to operation 204. At operation 204, the processor analyzes, statistically, the congregation data. In some embodiments, the method 200 proceeds to operation 206. At operation 206, the processor generates a social movement model of the particular area.

In some embodiments, the method 200 proceeds to operation 208. At operation 208, the processor generates one or more routes from a first location to a second location in the particular area based on the social movement model. In these embodiments, the one or more routes may reduce a user's level of exposure to the infectious disease. In some embodiments, as depicted in FIG. 2, after operation 208, the method 200 may end.

As discussed in more detail herein, it is contemplated that some or all of the operations of the method 200 may be performed in alternative orders or may not be performed at all; furthermore, multiple operations may occur at the same time or as an internal part of a larger process.

It is to be understood that although this disclosure includes a detailed description on cloud computing, implementation of the teachings recited herein are not limited to a cloud computing environment. Rather, embodiments of the present disclosure are capable of being implemented in conjunction with any other type of computing environment currently known or that which may be later developed.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of portion independence in that the consumer generally has no control or knowledge over the exact portion of the provided resources but may be able to specify portion at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

Referring now to FIG. 2, a flowchart illustrating an example method 200 for language translation during a conference call, in accordance with embodiments of the present disclosure. In some embodiments, the method 200 may be used to perform language morphing or language translation in real-time during a conference call.

In some embodiments, the method 200 begins at operation 202 where a processor initiates the conference call having a speaker and a listener. In some embodiments, the listener receives voice data from the speaker in a first language. The method 200 proceeds to operation 204. At operation 204, the processor determines a preferred language of the speaker and a preferred language of the listener. The method 200 proceeds to operation 206. At operation 206, the processor determines a fluency level of the speaker from the voice data of the first language. The method 200 proceeds to operation 208. At operation 208, the processor determines a difficulty level of the listener based, at least in part, on one or more attributes of the listener and the voice data received by the listener. The method 200 proceeds to operation 210. At operation 210, the processor exceeds an understanding threshold. In embodiments, the understanding threshold is based, at least in part, on the fluency level and the difficulty level. The method 200 proceeds to operation 212. At operation 212, the processor suggests, responsive to exceeding the understanding threshold, a translation option. In embodiments, the translation option may be based, on the preferred language of the speaker and the preferred language of the listener. In some embodiments, as depicted in FIG. 2, after operation 212, the method 200 may end.

In some embodiments, discussed below, there are one or more operations of the method 200 that were not depicted for the sake of brevity with operations/steps further performed by the processor.

Accordingly, in embodiments, the processor may determine the fluency level of the speaker by observing one or more vocal attributes in the voice data of the speaker, analyzing the one or more speaker attributes, comparing the one or more speaker attributes of the speaker to a historical repository of attributes, and generating an understanding of the one or more speaker attributes of the speaker.

In embodiments, the processor may suggest a translation option by indicating to the speaker to provide voice data in the speaker's preferred language. In these embodiments, the processor may capture the voice data of the speaker speaking the preferred language during the conference call. In these embodiments, the processor may translate the voice data of the speaker's preferred language to the listener's preferred language.

In embodiments the processor may determine the difficulty level of the listener by observing the one or more listener attributes of the listener during the conference call. The processor may also analyze the one or more listener attributes and compare the one or more listener attributes to a historical repository of attributes. In embodiments, the processor may generate an understanding of the one or more listener attributes of the listener.

In embodiments, a processor may suggest a translation option. In these embodiments, a processor may analyze the one or more listener attributes of the listener, responsive to the listener receiving the voice data of the speaker in the first language. In these embodiments, the processor may identify an expression from the voice data of the speaker in the first language. In embodiments, the processor may determine that the expression, using the one or more attributes and corresponding difficulty level, may cause the understanding threshold to be exceeded. In embodiments, the processor may indicate to the speaker to provide voice data associated with the expression in the speaker's preferred language. In embodiments, the processor may provide the speaker with a list of alternative expressions in the first language that correlate to the expression. In these embodiments, a list of alternative expressions may be one of the suggested translation options.

In embodiments, a processor may analyze the speaker's preferred language and the listener's preferred language and determine that the respective preferred languages of the listener and speaker are different.

In embodiments, a processor may analyze the first language spoken by the speaker and the listener's preferred language are the same language.

In embodiments, a processor can select a translation option from a group consisting of controlled languages, and constructed languages.

In embodiments, a processor may display a closed caption, wherein the closed caption is a translation of the voice data from the speaker in the first language to the listener's preferred language.

In embodiments, a processor may display a closed caption of the voice data from the speaker in a first language in multiple languages.

In embodiments, a processor may analyze the conference call and identify a new/unknown listener in an operating zone of the conference call. A processor may determine whether the unknown listener is an unauthorized listener. In these embodiments, a processor may determine whether the unknown listener is an unauthorized listener by analyzing one or more historical patterns of the conference call to determine if the unknown listener has authorization. In these embodiments, a processor may further analyze the voice data associated with the conference call to determine if the voice data contains one or more pieces of confidential information. In embodiments, responsive to determining the unknown listener is an unauthorized listener, a processor may switch the conference call to a confidential mode.

A cloud computing environment is service oriented with a focus on statelessness, low coupling, modularity, and semantic interoperability. At the heart of cloud computing is an infrastructure that includes a network of interconnected nodes.

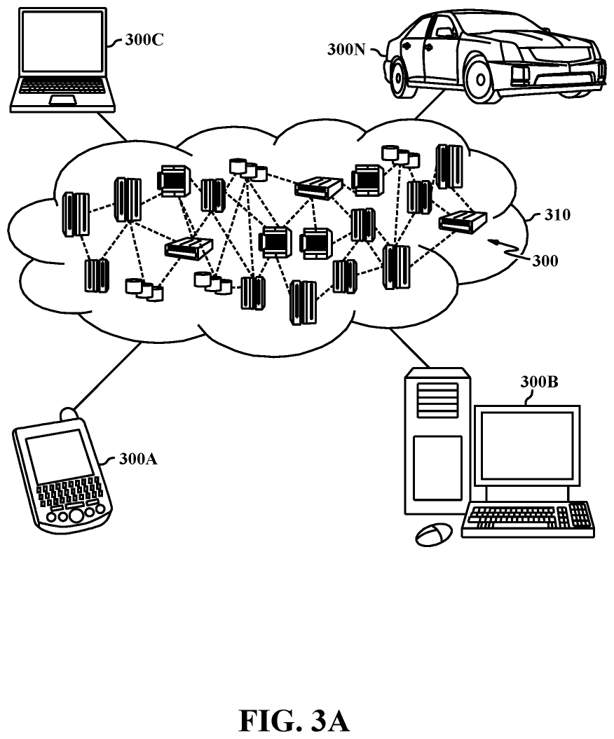

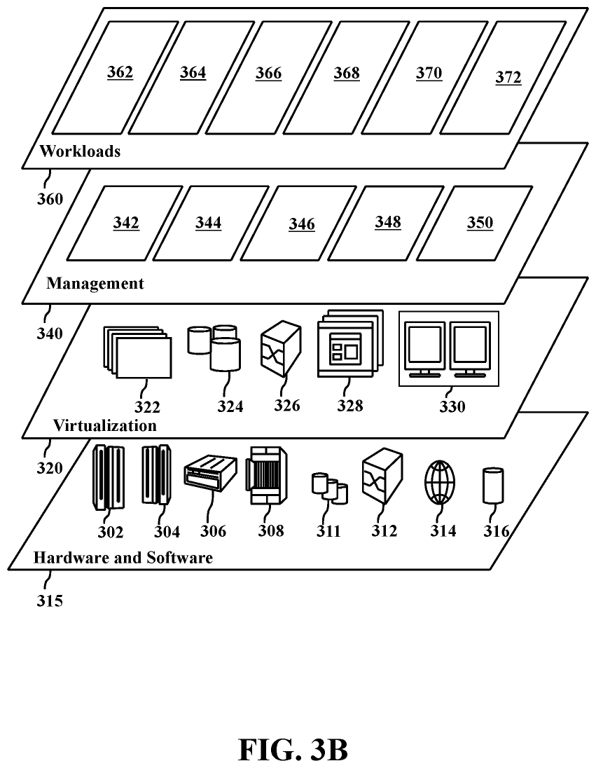

Referring now to FIG. 3A, illustrative cloud computing environment 310 is depicted. As shown, cloud computing environment 310 includes one or more cloud computing nodes 300 with which local computing devices used by cloud consumers, such as, for example, personal digital assistant (PDA) or cellular telephone 300A, desktop computer 300B, laptop computer 300C, and/or automobile computer system 300N may communicate. Nodes 300 may communicate with one another. They may be grouped (not shown) physically or virtually, in one or more networks, such as Private, Community, Public, or Hybrid clouds as described hereinabove, or a combination thereof. This allows cloud computing environment 310 to offer infrastructure, platforms and/or software as services for which a cloud consumer does not need to maintain resources on a local computing device. It is understood that the types of computing devices 300A-N shown in FIG. 3A are intended to be illustrative only and that computing nodes 300 and cloud computing 300 and cloud computing environment 310 can communicate with any type of computerized device over any type of network and/or network addressable connection (e.g., using a web browser).

Referring now to FIG. 3B, a set of functional abstraction layers provided by cloud computing environment 310 (FIG. 3A) is shown. It should be understood in advance that the components, layers, and functions shown in FIG. 3B are intended to be illustrative only and embodiments of the disclosure are not limited thereto. As depicted below, the following layers and corresponding functions are provided.

Hardware and software layer 315 include hardware and software components. Examples of hardware components include: mainframes 302; RISC (Reduced Instruction Set Computer) architecture based servers 304; servers 306; blade servers 308; storage devices 311; and networks and networking components 312. In some embodiments, software components include network application server software 314 and database software 316.

Virtualization layer 320 provides an abstraction layer from which the following examples of virtual entities may be provided: virtual servers 322; virtual storage 324; virtual networks 326, including virtual private networks; virtual applications and operating systems 328; and virtual clients 330.

In one example, management layer 340 may provide the functions described below. Resource provisioning 342 provides dynamic procurement of computing resources and other resources that are utilized to perform tasks within the cloud computing environment. Metering and Pricing 344 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 346 provides access to the cloud computing environment for consumers and system administrators. Service level management 348 provides cloud computing resource allocation and management such that required service levels are met. Service Level Agreement (SLA) planning and fulfillment 350 provide pre-arrangement for, and procurement of, cloud computing resources for which a future requirement is anticipated in accordance with an SLA.

Workloads layer 360 provides examples of functionality for which the cloud computing environment may be utilized. Examples of workloads and functions which may be provided from this layer include: mapping and navigation 362; software development and lifecycle management 364; virtual classroom education delivery 366; data analytics processing 368; transaction processing 370; and social distancing optimization 372.

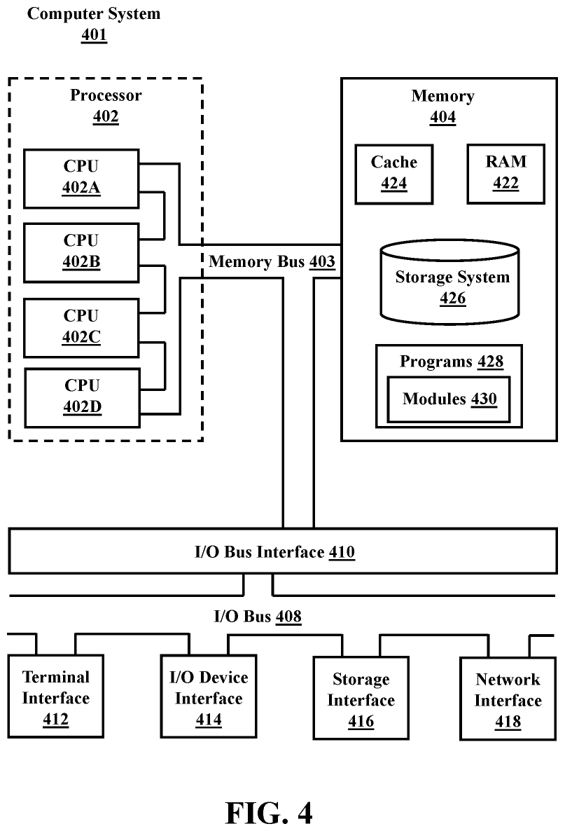

FIG. 4, illustrated is a high-level block diagram of an example computer system 401 that may be used in implementing one or more of the methods, tools, and modules, and any related functions, described herein (e.g., using one or more processor circuits or computer processors of the computer), in accordance with embodiments of the present invention. In some embodiments, the major components of the computer system 401 may comprise one or more Processor 402, a memory subsystem 404, a terminal interface 412, a storage interface 416, an I/O (Input/Output) device interface 414, and a network interface 418, all of which may be communicatively coupled, directly or indirectly, for inter-component communication via a memory bus 403, an I/O bus 408, and an I/O bus interface unit 410.

The computer system 401 may contain one or more general-purpose programmable central processing units (CPUs) 402A, 402B, 402C, and 402D, herein generically referred to as the CPU 402. In some embodiments, the computer system 401 may contain multiple processors typical of a relatively large system; however, in other embodiments the computer system 401 may alternatively be a single CPU system. Each CPU 402 may execute instructions stored in the memory subsystem 404 and may include one or more levels of on-board cache.

System memory 404 may include computer system readable media in the form of volatile memory, such as random access memory (RAM) 422 or cache memory 424. Computer system 401 may further include other removable/non-removable, volatile/non-volatile computer system storage media. By way of example only, storage system 426 can be provided for reading from and writing to a non-removable, non-volatile magnetic media, such as a “hard drive.” Although not shown, a magnetic disk drive for reading from and writing to a removable, non-volatile magnetic disk (e.g., a “floppy disk”), or an optical disk drive for reading from or writing to a removable, non-volatile optical disc such as a CD-ROM, DVD-ROM or other optical media can be provided. In addition, memory 404 can include flash memory, e.g., a flash memory stick drive or a flash drive. Memory devices can be connected to memory bus 403 by one or more data media interfaces. The memory 404 may include at least one program product having a set (e.g., at least one) of program modules that are configured to carry out the functions of various embodiments.

One or more programs/utilities 428, each having at least one set of program modules 430 may be stored in memory 404. The programs/utilities 428 may include a hypervisor (also referred to as a virtual machine monitor), one or more operating systems, one or more application programs, other program modules, and program data. Each of the operating systems, one or more application programs, other program modules, and program data or some combination thereof, may include an implementation of a networking environment. Programs 428 and/or program modules 430 generally perform the functions or methodologies of various embodiments.

Although the memory bus 403 is shown in FIG. 4 as a single bus structure providing a direct communication path among the CPUs 402, the memory subsystem 404, and the I/O bus interface 410, the memory bus 403 may, in some embodiments, include multiple different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface 410 and the I/O bus 408 are shown as single respective units, the computer system 401 may, in some embodiments, contain multiple I/O bus interface units 410, multiple I/O buses 408, or both. Further, while multiple I/O interface units are shown, which separate the I/O bus 408 from various communications paths running to the various I/O devices, in other embodiments some or all of the I/O devices may be connected directly to one or more system I/O buses.

In some embodiments, the computer system 401 may be a multi-user mainframe computer system, a single-user system, or a server computer or similar device that has little or no direct user interface, but receives requests from other computer systems (clients). Further, in some embodiments, the computer system 401 may be implemented as a desktop computer, portable computer, laptop or notebook computer, tablet computer, pocket computer, telephone, smartphone, network switches or routers, or any other appropriate type of electronic device.

It is noted that FIG. 4 is intended to depict the representative major components of an exemplary computer system 401. In some embodiments, however, individual components may have greater or lesser complexity than as represented in FIG. 4, components other than or in addition to those shown in FIG. 4 may be present, and the number, type, and configuration of such components may vary.

As discussed in more detail herein, it is contemplated that some or all of the operations of some of the embodiments of methods described herein may be performed in alternative orders or may not be performed at all; furthermore, multiple operations may occur at the same time or as an internal part of a larger process.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Although the present invention has been described in terms of specific embodiments, it is anticipated that alterations and modification thereof will become apparent to the skilled in the art. Therefore, it is intended that the following claims be interpreted as covering all such alterations and modifications as fall within the true spirit and scope of the disclosure.

What is claimed is:

1. A method for reducing a level of exposure of contact with an infectious disease, the method comprising:
receiving, by a processor, congregation data, wherein congregation data is associated with a particular area;
analyzing the congregation data;
generating a social movement model of the particular area; and
generating one or more routes from a first location to a second location in the particular area based on the social movement model, wherein the one or more routes reduce the level of exposure to the infectious disease.

2. The method of claim 1, wherein the congregation data is received from one or more devices belonging to one or more users in the particular area

3. The method of claim 1, wherein generating the social movement model includes:
analyzing regional data of the particular area.

4. The method of claim 1, wherein analyzing the congregation data includes:
constructing a sparsity grid of the particular area;
performing a queue analysis on the congregation data; and
modeling one or more random walks based, at least in part, on the congregation data.

5. The method of claim 1, wherein generating the social movement model of the particular area further includes:
producing a matrix using a set of input feeds, wherein the set of input feeds are based on the results of the congregation data that was statistically analyzed.

6. The method of claim 5, wherein generating the social movement model of the particular area further includes:
overlaying the matrix over a sparsity grid of the particular area;

7. The method of claim 1, wherein generating the social movement model of the particular area includes:
determining if at least one user from a group of two or more users are likely located in the particular area.

8. The method of claim 1, further comprising:
generating a predicted level of exposure associated with each of the one or more routes.

9. The method of claim 1, further comprising:
choosing a particular route from the one or more routes;
receiving travel data associated with travel from the first location to the second location; and
generating an actual level of exposure of the infectious disease.

10. A system for reducing a level of exposure of contact with an infectious disease, the system comprising:
a memory; and
a processor in communication with the memory, the processor being configured to perform operations comprising:
receiving congregation data, wherein congregation data is associated with a particular area;
analyzing the congregation data;
generating a social movement model of the particular area; and
generating one or more routes from a first location to a second location in the particular area based on the social movement model, wherein the one or more routes reduce the level of exposure to the infectious disease.

11. The system of claim 10, wherein the congregation data is received from one or more devices belonging to one or more users in the particular area

12. The system of claim 10, wherein generating the social movement model includes:
analyzing regional data of the particular area.

13. The system of claim 10, wherein analyzing the congregation data includes:
constructing a sparsity grid of the particular area;
performing a queue analysis on the congregation data; and
modeling one or more random walks based, at least in part, on the congregation data.

14. The system of claim 10, wherein generating the social movement model of the particular area further includes:
producing a matrix using a set of input feeds, wherein the set of input feeds are based on the results of the congregation data that was statistically analyzed.

15. The system of claim 14, wherein generating the social movement model of the particular area further includes:
overlaying the matrix over a sparsity grid of the particular area;

16. The system of claim 10, wherein generating the social movement model of the particular area includes:
determining if at least one user from a group of two or more users are likely located in the particular area.

17. The system of claim 10, wherein the operations further comprise:
generating a predicted level of exposure associated with each of the one or more routes.

18. The system of claim 10, wherein the operations further comprise:
choosing a particular route from the one or more routes;
receiving travel data associated with travel from the first location to the second location; and
generating an actual level of exposure of the infectious disease.

19. A computer program product for reducing a level of exposure of contact with an infectious disease, the computer program product comprising a computer readable storage medium having program instructions embodied therewith, the program instructions executable by a processor to cause the processors to perform a function, the function comprising:
receiving congregation data, wherein congregation data is associated with a particular area;
analyzing the congregation data;
generating a social movement model of the particular area; and
generating one or more routes from a first location to a second location in the particular area based on the social movement model, wherein the one or more routes reduce the level of exposure to the infectious disease.

20. The computer program product of claim 19, wherein the congregation data is received from one or more devices belonging to one or more users in the particular area

21. The computer program product of claim 19, wherein generating the social movement model includes:
analyzing regional data of the particular area.

22. The computer program product of claim 19, wherein analyzing the congregation data includes:
constructing a sparsity grid of the particular area;
performing a queue analysis on the congregation data; and
modeling one or more random walks based, at least in part, on the congregation data.

23. The computer program product of claim 19, wherein generating the social movement model of the particular area further includes:
producing a matrix using a set of input feeds, wherein the set of input feeds are based on the results of the congregation data that was statistically analyzed.

24. The computer program product of claim 23, wherein generating the social movement model of the particular area further includes:
overlaying the matrix over a sparsity grid of the particular area;

25. The computer program product of claim 19, wherein generating the social movement model of the particular area includes:
determining if at least one user from a group of two or more users are likely located in the particular area.
